(19) United States
(12) Patent Application Publication
(10) Pub. No.: US 2026/0021296 A1
(43) Pub. Date: Jan. 22, 2026

(54) IONTOPHORESIS DEVICE

(71) Applicant: KANEKA CORPORATION, Osaka (JP)

(72) Inventors: Mirai Tanaka, Hyogo (JP); Hiroyuki Ogino, Hyogo (JP); Masaoki Goto, Hyogo (JP)

(73) Assignee: KANEKA CORPORATION, Osaka (JP)

(21) Appl. No.: 19/339,658

(22) Filed: Sep. 25, 2025

Related U.S. Application Data
(63) Continuation of application No. PCT/JP2024/011217, filed on Mar. 22, 2024.

(30) Foreign Application Priority Data
Mar. 30, 2023 (JP) 2023-055104

(51) Int. Cl. A61N 1/32; A61N 1/04; A61N 1/08

(52) U.S. Cl. CPC A61N 1/325; A61N 1/0448; A61N 1/08

(57) ABSTRACT

An iontophoresis device capable of reducing skin irritability due to an applied current while enhancing skin permeability of a drug is provided. The iontophoresis device includes a power supply, an electrode electrically connected to the power supply, a drug disposed between skin and the electrode, and a control unit configured to control a current applied to the skin via the electrode. The control unit controls the current so that a pulse current having a pulse frequency of 0.5 Hz or more and 50 Hz or less is applied to the skin.

### TECHNICAL FIELD

One or more embodiments of the present invention relate to an iontophoresis device.

### BACKGROUND

In the related art, iontophoresis is known in which a drug disposed between an electrode and the skin is supplied into the body through the skin by applying a current or a voltage to the skin through the electrode. Iontophoresis devices are used to perform iontophoresis.

Patent Literature 1 discloses an iontophoresis device that uses a pulse current having a pulse width in the range of 50 us to 100 ms, a pulse frequency in the range of 1 Hz to 200 Hz, and a duty ratio in the range of 1% to 90% to increase the skin permeability of drugs. Patent Literature 2 discloses an electrotransport agent delivery device that uses a pulse current having a pulse width of 5 ms or more, a pulse frequency of less than 10 Hz, and a duty ratio in the range of 30% to 90% to increase the skin permeability of drugs. Patent Literature 3 discloses a configuration that applies a combination of pulse depolarization-type current and pulse-type current to provide a transmucosal drug delivery device with excellent drug skin permeability and reduced skin irritability.

### PATENT LITERATURE

PTL 1: Japanese Unexamined Patent Application Publication (Translation of PCT Application) No. 2020-503349
PTL 2: Japanese Unexamined Patent Application Publication (Translation of PCT Application) No. H11-507280
PTL 3: International Publication No. 1999/000157

When a current is applied to the skin by an iontophoresis device, skin irritation such as erythema may occur in a portion of the skin to which the current is applied. However, Patent Literature 1 and Patent Literature 2 do not disclose such skin irritability caused by current application. The configuration described in Patent Literature 3 may not be able to reduce skin irritability.

### SUMMARY

One or more embodiments of the present invention are to reduce skin irritability due to an applied current while enhancing skin permeability of a drug.

An iontophoresis device according to one or more embodiments of the present invention includes a power supply, an electrode electrically connected to the power supply, a drug disposed between skin and the electrode, and a control unit configured to control a current applied to the skin via the electrode, in which the control unit performs control so that a pulse current having a pulse frequency of 0.5 Hz or more and 50 Hz or less is applied to the skin.

For example, skin irritability can be reduced by the following iontophoresis devices.
[1] An iontophoresis device including:
an electrode attached to skin via a drug; and
a control unit that is connected to the electrode and configured to generate a pulse current having a frequency of 0.5 Hz or more and 50 Hz or less.
[2] An iontophoresis device including:
a power supply;
an electrode electrically connected to the power supply;
a drug disposed between skin and the electrode; and
a control unit configured to control a current applied to the skin via the electrode,
wherein the control unit performs control so that a pulse current having a pulse frequency of 0.5 Hz or more and 50 Hz or less is applied to the skin.
[3] The iontophoresis device according to item [1], wherein the pulse current has a pulse width of 10 ms or more and 1000 ms or less.
[4] The iontophoresis device according to item [2], wherein the pulse current has a pulse width of 10 ms or more and 1000 ms or less.
[5] An iontophoresis device including:
an electrode attached to skin via a drug; and
a control unit that is connected to the electrode and configured to generate a pulse current having a pulse width of 10 ms or more and 1000 ms or less.
[6] An iontophoresis device including:
a power supply;
an electrode electrically connected to the power supply;
a drug disposed between skin and the electrode; and
a control unit configured to control a current applied to the skin via the electrode,
wherein the control unit performs control so that a pulse current having a pulse width of 10 ms or more and 1000 ms or less is applied to the skin.
[7] The iontophoresis device according to any one of items [1], [3], and [5], wherein the control unit generates the pulse current by applying a pulse voltage subjected to constant voltage control to a constant current element.
[8] The iontophoresis device according to any one of items [2], [4], and [6], wherein the control unit generates the pulse current by applying a pulse voltage subjected to constant voltage control to a constant current element.
[9] The iontophoresis device according to any one of items [1], [3], [5], and [7],
wherein the electrode includes a pair of electrodes including a first electrode and a second electrode, and
the iontophoresis device includes a first reservoir that is disposed between the skin and the first electrode and stores the drug, and
a second reservoir that is disposed between the skin and the second electrode and stores an electrolyte for moving ions.
[10] The iontophoresis device according to any one of items [2], [4], [6], and [8],
wherein the electrode includes a pair of electrodes including a first electrode and a second electrode,
the iontophoresis device includes a first reservoir that is disposed between the skin and the first electrode and stores the drug, and
a second reservoir that is disposed between the skin and the second electrode and stores an electrolyte for moving ions, and
the control unit controls the current applied to the skin via the first electrode.

According to one or more embodiments of the present invention, it is possible to reduce skin irritability due to an applied current while increasing skin permeability of a drug.

### BRIEF DESCRIPTION OF THE DRAWINGS

FIG. 1A is a view showing a state in which a first example of iontophoresis according to one or more embodiments is attached to the skin.

FIG. 1B is a view showing a state in which a second example of iontophoresis according to one or more embodiments is attached to the skin.

FIG. 2 is an enlarged view schematically showing region II in FIG. 1A and FIG. 1B.

FIG. 3 is an exploded perspective view schematically showing an overall configuration example of an iontophoresis device according to one or more embodiments.

FIG. 4A is a top view schematically showing a first example of the overall configuration of the iontophoresis device according to one or more embodiments.

FIG. 4B is a top view schematically showing a second example of the overall configuration of the iontophoresis device according to one or more embodiments.

FIG. 5A is a schematic cross-sectional view taken along line VA-VA in FIG. 4A.

FIG. 5B is a schematic cross-sectional view taken along line VB-VB in FIG. 4B.

FIG. 6 is a block diagram showing a configuration example of a control unit according to one or more embodiments.

FIG. 7 is a diagram for explaining an example of a pulse current according to one or more embodiments.

FIG. 8 is a graph showing an example of the cumulative amount of drug permeated.

FIG. 9 is a graph showing an example of the permeation rate of a drug.

FIG. 10 is a graph showing an example of the relationship between the cumulative amount of drug permeated and the quantity of electricity.

### DETAILED DESCRIPTION

Hereinafter, one or more embodiments for carrying out the invention will be described with reference to the drawings. In the drawings, the same components are denoted by the same reference numerals, and redundant description thereof will be appropriately omitted.

The embodiments described below are examples of an iontophoresis device for embodying the technical idea of one or more embodiments of the present invention, and the present invention is not limited to the embodiments described below. Unless otherwise specified, dimensions, materials, shapes, relative arrangements, and the like of components described below are not intended to limit the scope of the present invention thereto, but are intended to exemplify one or more embodiments of the present invention. In addition, the size, positional relationship, and the like of members shown in the drawings may be exaggerated for clarity of description.

In some of the drawings described below, an orthogonal coordinate system including an X axis, a Y axis, and a Z axis is used as a direction representation. The X axis, the Y axis, and the Z axis are substantially orthogonal to each other. The Z direction along the Z axis is defined as a direction along the normal direction of the skin to which an iontophoresis device according to one or more embodiments is attached. A direction toward the skin is defined as a −Z direction, and a direction away from the skin is defined as a +Z direction. In this specification, the −Z direction is referred to as “down”, and the +Z direction is referred to as “up”. In addition, the top view refers to viewing an object from the +Z direction side. However, these direction representations do not limit the direction in one or more embodiments, and the orientation of the iontophoresis device according to one or more embodiments during use is arbitrary. In this specification, the term “along” can be replaced with “substantially parallel.” In this specification, “substantially parallel” means that a deviation of ±10 degrees or less with respect to parallel may be included. Also, in this specification, “substantially orthogonal” means that a deviation of ±10 degrees or less with respect to orthogonal may be included.

In the present specification and claims, the term “drug” includes a “medicine” including a dosage form and a “drug solution” obtained by dissolving a drug into a liquid form.

### <Examples of Method of Using Iontophoresis Device 100>

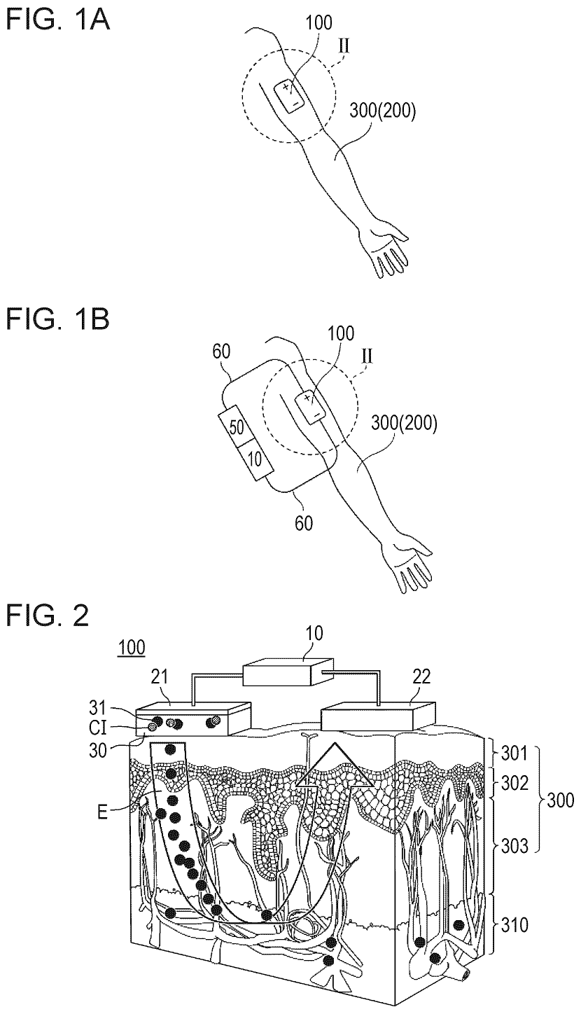

First, a method of using an iontophoresis device 100 according to one or more embodiments will be described with reference to FIGS. 1A, 1B, and 2. FIGS. 1A and 1B are views showing a state in which the iontophoresis device 100 is attached to skin 300 of a living body 200. FIG. 1A shows a case where a power supply and a control unit are built into the device, and FIG. 1B shows a case where a power supply and a control unit are installed externally. FIG. 2 is an enlarged view schematically showing region II in FIG. 1A and FIG. 1B.

Iontophoresis is a technique in which a current or voltage is applied to the skin through an electrode, and a drug placed between the electrode and the skin is delivered into the body through the skin by electrical repulsion due to the applied current or applied voltage, or by a convective movement of water such as electroosmotic flow. Examples of drugs transdermally delivered by iontophoresis include low-molecular-weight ionic drugs, macromolecular drugs such as peptides, proteins, and oligonucleotides, as well as antibody drugs and nucleic acid drugs.

The iontophoresis device 100 is a device used to perform iontophoresis. As shown in FIG. 1A and FIG. 1B, the iontophoresis device 100 is used while being attached to the surface of the skin 300 of the living body 200. FIG. 1B illustrates an example of a state in which the components of the iontophoresis device 100 other than a power supply 10 and a control unit 50 are attached to the skin 300 of the living body 200, and the power supply 10 and the control unit 50 of the iontophoresis device 100 are disposed at a position other than the skin 300. The components of the iontophoresis device 100 other than the power supply 10 and the control unit 50 are electrically connected to the power supply 10 and the control unit 50 of the iontophoresis device 100 via wiring lines 60. As shown in FIG. 2, the skin 300 is configured to include stratum corneum 301, epidermis 302, dermis 303, and the like in order from the surface. Subcutaneous tissue 310 is present inside the dermis 303.

The iontophoresis device 100 applies a current to the skin 300 via an anode 21 serving as an electrode by the power supply 10. A drug 31 stored in a first reservoir 30 is disposed between the anode 21 and the skin 300. The drug 31 reaches the subcutaneous tissue 310 via the skin 300 by electrical repulsion according to an applied current E and an action of the convective movement of water. The drug 31 is supplied to various parts within the living body through the subcutaneous tissue 310. In FIG. 2, the drug 31 is indicated by black circles. Circles indicated by dot hatching each indicate a counterion CI of the drug 31.

As described above, the iontophoresis device 100 can supply the drug 31 into the living body.

### <Configuration Examples of Iontophoresis Device 100>

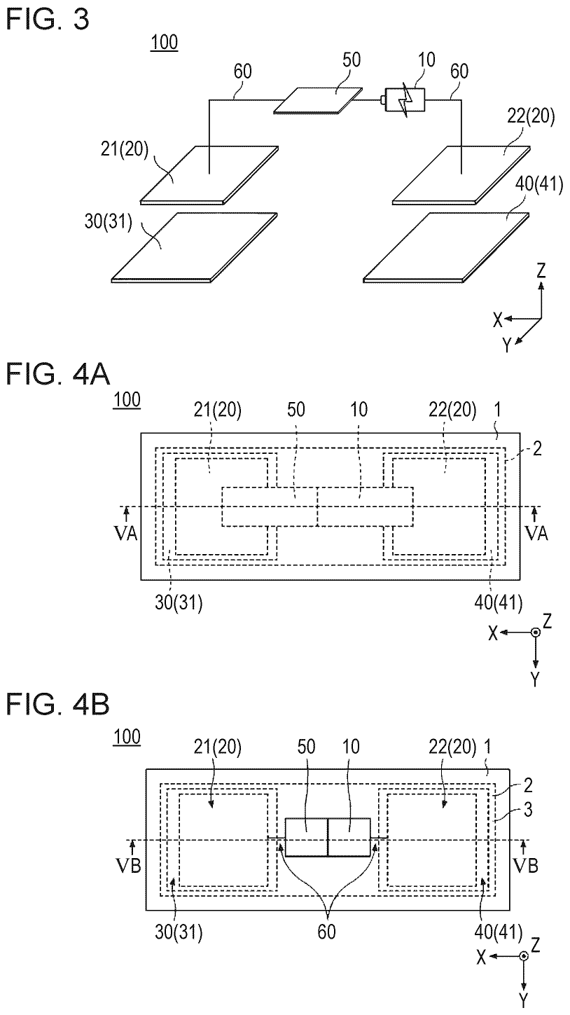

The configuration of the iontophoresis device 100 will be described in detail with reference to FIGS. 3 to 6.

### (Overall Configuration)

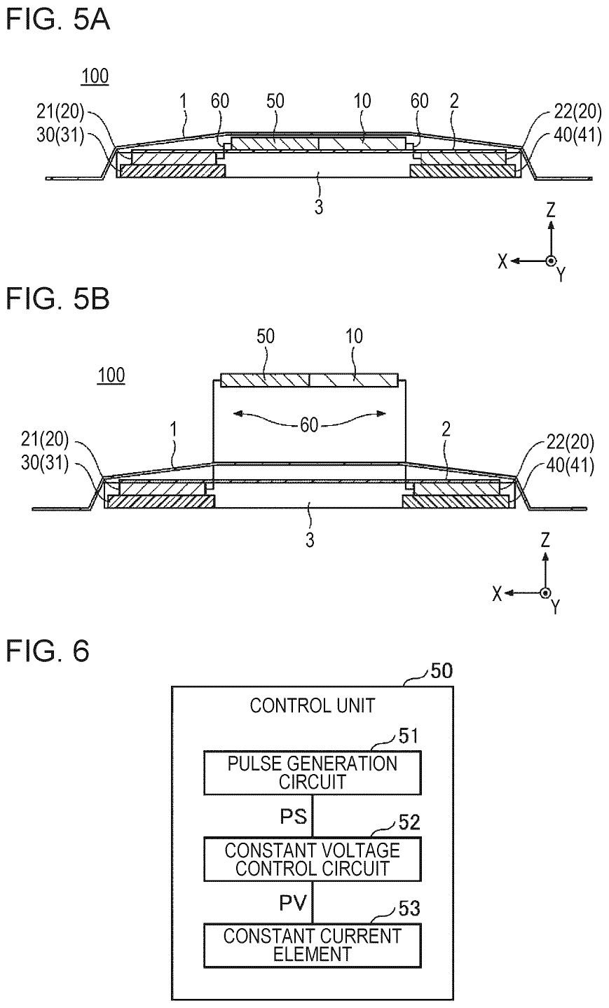

FIGS. 3 to 5B are views that schematically show an example of the overall configuration of the iontophoresis device 100. FIG. 3 is an exploded perspective view. FIG. 4A is a top view of a first example. FIG. 4B is a top view of a second example. FIG. 5A is a cross-sectional view taken along line VA-VA in FIG. 4A. FIG. 5B is a cross-sectional view taken along line VB-VB in FIG. 4B. FIG. 4A and FIG. 5A show a case where the power supply and the control unit are built into the device, and FIG. 4B and FIG. 5B show a case where the power supply and the control unit are installed externally.

As shown in FIGS. 3 to 5B, the iontophoresis device 100 includes a power supply 10, a pair of electrodes 20, a drug 31, a first reservoir 30, a second reservoir 40, and a control unit 50. The pair of electrodes 20 includes an anode 21 and a cathode 22. As shown in FIG. 3, the anode 21 is electrically connected to the positive electrode of the power supply 10 via the control unit 50, and the cathode 22 is electrically connected to the negative electrode of the power supply 10, both by wiring lines 60. However, the method of electrically connecting the power supply 10, the anode 21, and the cathode 22 together is not limited to using the wiring lines 60, and can be appropriately changed according to usage conditions, etc. The wiring lines 60 may each be in the form of a thin film, a plate, a cable, or the like. For example, a printed wiring line produced by screen printing, etching, or the like can be used as the thin film, a metal plate or the like can be used as the plate, and a coated metal wire or the like can be used as the cable. In addition, the control unit 50 is not necessarily connected between the anode 21 and the positive electrode of the power supply 10, and may be disposed between the cathode 22 and the negative electrode of the power supply 10. In addition, a method of electrically connecting the power supply 10, the control unit 50, the anode 21, and the cathode 22 together is not limited to wired connection, and may be wireless connection using radio waves or the like. For example, at least one of the power supply 10 and the control unit 50 in the iontophoresis device 100 may be disposed at a position other than the skin 300 of the living body 200, and the components other than at least one of the power supply 10 and the control unit 50 in the iontophoresis device 100 may be disposed on the skin. At least one of the power supply 10 and the control unit 50 may be wirelessly connected to the components other than the at least one of the power supply 10 and the control unit 50 so that they can communicate with each other. In other words, the iontophoresis device 100 may have an integrated configuration in which all components are attached to the surface of the skin 300. Alternatively, the iontophoresis device 100 may have a separated configuration in which some components included in the iontophoresis device 100 are placed on the skin 300 of the living body 200, while other components included in the iontophoresis device 100 are positioned away from the living body 200.

As shown in FIGS. 4A to 5B, a base material 2 supports at least the anode 21 and the cathode 22. The base material 2 is a plate-like member having a substantially rectangular outer shape in a top view. The base material 2 supports the anode 21 and the cathode 22 by bonding them to its lower surface with adhesive members or the like. In FIG. 4A and FIG. 5A, the base material 2 further supports the power supply 10 and the control unit 50 by bonding them to its upper surface with adhesive members or the like. The wiring lines 60 may be supported by the base material 2. For the material of the base material 2, an insulator can be used. For example, a resin film or sheet, paper material, cloth material, or the like can be used. The outer shape of the base material 2 in a top view is not limited to a substantially rectangular shape, and may be a substantially circular shape, a substantially elliptical shape, a substantially polygonal shape, a substantially dumbbell shape, or the like. The iontophoresis device 100 may not necessarily include the base material 2.

The anode 21 supports the first reservoir 30 by bonding it to its lower surface with an adhesive member or the like. The cathode 22 supports the second reservoir 40 by bonding it to its lower surface with an adhesive member or the like. In other words, the base material 2 supports the first reservoir 30 via the anode 21 and supports the second reservoir 40 via the cathode 22.

In FIGS. 4A to 5B, a skin contact member 3 supports the base material 2 by bonding the base material 2 to its upper surface with an adhesive member or the like. In addition, the skin contact member 3 fixes the iontophoresis device 100 to the surface of the skin 300 by adhering it to its lower surface with a pressure-sensitive adhesive member or the like, and fixes the positions of the first reservoir 30 and the second reservoir 40 when the iontophoresis device 100 is fixed to the surface of the skin 300. The skin contact member 3 has holes with diameters equal to or larger than those of the first reservoir 30 and the second reservoir 40, and the positions of the first reservoir 30 and the second reservoir 40 are fixed by installing the first reservoir 30 and the second reservoir 40 in the holes. The shape of each of the holes is substantially rectangular, substantially circular, substantially elliptical, substantially polygonal, or the like. As a material of the skin contact member 3, an insulator can be used. For example, a foam or sheet made of resin or rubber, paper material, cloth material, or the like having sufficient thickness and conformability to the skin can be used. The iontophoresis device 100 does not necessarily need to include the skin contact member 3.

A cover tape 1 in FIGS. 4A to 5B is a member having adhesiveness on the lower surface. The cover tape 1 bonds or adheres the power supply 10, the pair of electrodes 20, the drug 31, the first reservoir 30, the second reservoir 40, and the control unit 50, which are supported by the base material 2 and the skin contact member 3, to a part of the lower surface, while the other part of the lower surface is adhered to the surface of the skin 300 (see FIGS. 1A to 2). Thus, the cover tape 1 can fix the power supply 10, the pair of electrodes 20, the drug 31, the first reservoir 30, the second reservoir 40, and the control unit 50, which are supported by the base material 2 and the skin contact member 3, to the surface of the skin 300. Note that, in the iontophoresis device 100, the power supply 10, the pair of electrodes 20, the drug 31, the first reservoir 30, the second reservoir 40, and the control unit 50 may be fixed to the surface of the skin 300 by means other than the cover tape 1.

Also, as shown in FIG. 4B and FIG. 5B, at least a part of the cover tape 1, the base material 2, the skin contact member 3, the anode 21, the cathode 22, the first reservoir 30, and the second reservoir 40 is attached to the skin 300. The power supply 10 and the control unit 50 may be positioned and used at a location away from the user via the wiring lines 60 connected to the anode 21 and the cathode 22.

In FIGS. 3 to 5B, a primary battery, a secondary battery, etc. can be used as the power supply 10. For example, the power supply 10 may include two directly connected coin-type lithium batteries to generate a maximum voltage of 6 V. From the viewpoint of reducing skin irritability, the current density may be 0.4 mA/cm² or less. Note that skin irritation refers to changes in skin properties such as erythema occurring in portions of the skin 300 to which a current is applied. Skin irritability refers to the property that causes skin irritation.

When using the iontophoresis device 100 as a single-use application, it is preferable to use an inexpensive primary battery such as a coin-type lithium battery as the power supply 10 in order to reduce costs. The single-use application refers to when one iontophoresis device 100 is used for a single drug delivery to a living body and then discarded.

The anode 21 corresponds to an electrode electrically connected to the power supply 10 and corresponds to a first electrode. The cathode 22 corresponds to a second electrode. In FIGS. 3 to 5B, in the sense that the pair of electrodes 20 includes the anode 21 and the cathode 22, the reference numeral of the pair of electrodes 20 is written in parentheses together with the reference numerals of the anode 21 and the cathode 22. The materials of the anode 21 and the cathode 22 can be appropriately selected according to characteristics such as the ionized state of the drug 31 to be supplied. The anode 21 may contain a material such as silver, zinc, gold, platinum, titanium, or carbon from the viewpoint of biocompatibility, or may contain silver or zinc from the viewpoint of reducing pH change. The cathode 22 may contain, for example, silver/silver chloride, gold, platinum, titanium, or carbon from the viewpoint of biocompatibility, or may contain silver/silver halide from the viewpoint of reducing pH change. Examples of the halide salt constituting the silver/silver halide may include iodides, bromides, chlorides, and fluorides, or chlorides.

The areas of the anode 21 and the cathode 22 may each be 1 cm² or more and 50 cm² or less, 2 cm² or more and 25 cm² or less, or 3 cm² or more and 15 cm² or less.

The first reservoir 30 is disposed between the skin 300 located below the first reservoir 30 and the anode 21, and stores the drug 31. In FIGS. 3 to 5B, in the sense that the drug 31 is stored inside the first reservoir 30, the reference numeral of the drug 31 is written in parentheses together with the reference numeral of the first reservoir 30. The first reservoir 30 is configured to include a cotton nonwoven fabric pad or the like. The first reservoir 30 can store the drug 31 by immersing the cotton nonwoven fabric pad in a drug solution serving as the drug 31. Examples of the solvent used for immersion in the drug solution include buffer solutions and electrolyte solutions. As buffer solutions, for example, buffer solutions composed of acetic acid, phosphoric acid, citric acid, carbonic acid, or the like are used. As electrolytes, halide salts such as calcium chloride, potassium chloride, sodium chloride, etc. are used. The first reservoir 30 is not limited to the configuration including a cotton nonwoven fabric pad, and may be configured to include a porous membrane or a hydrogel. As porous membranes, foamed or porous polymer sheets or films, paper materials, cloth materials, etc. are used. As materials for these porous membranes, fluorocarbon polymers such as polytetrafluoroethylene, polyimide, polyolefins such as polyethylene, polyesters such as polyethylene terephthalate, cellulose polymers such as hydroxypropyl cellulose, silicone polymers such as polydimethylsiloxane, and the like are used. As the hydrogel, a hydrogel made of a resin derived from a natural product or a synthetic resin is used. Examples of the resin derived from a natural product include polysaccharides such as alginic acid, hyaluronic acid, and chitosan, metal salts thereof, and cellulose-based polymers such as carboxymethyl cellulose and hydroxypropyl cellulose. Examples of the synthetic resin include polyvinyl alcohol, polyvinyl pyrrolidone, polyvinyl methyl ether, polyacrylic acid and metal salts thereof, polyacrylamide and hydrolysates thereof, polyethers such as polyethylene glycol, and polymers having both a siloxane structure and an ether or ester structure in the molecular structure. The iontophoresis device 100 may not necessarily include the first reservoir 30. For example, the user of the iontophoresis device 100 may prepare the first reservoir 30.

The second reservoir 40 is disposed between the skin 300 located below the second reservoir 40 and the cathode 22, and stores an electrolyte 41 for moving ions. In FIGS. 3 to 5B, in the sense that the electrolyte 41 is stored inside the second reservoir 40, the reference numeral of the electrolyte 41 is written in parentheses together with the reference numeral of the second reservoir 40. The second reservoir 40 is configured to include, for example, a cotton nonwoven fabric pad. The second reservoir 40 can store the electrolyte 41 by immersing the cotton nonwoven fabric pad in a solution in which the electrolyte 41 is dissolved in a liquid. The second reservoir 40 is not limited to the configuration including the cotton nonwoven fabric pad, and may be configured to include a conductive gel containing the electrolyte 41. The iontophoresis device 100 may not necessarily include the second reservoir 40. For example, the user of the iontophoresis device 100 may prepare the second reservoir 40.

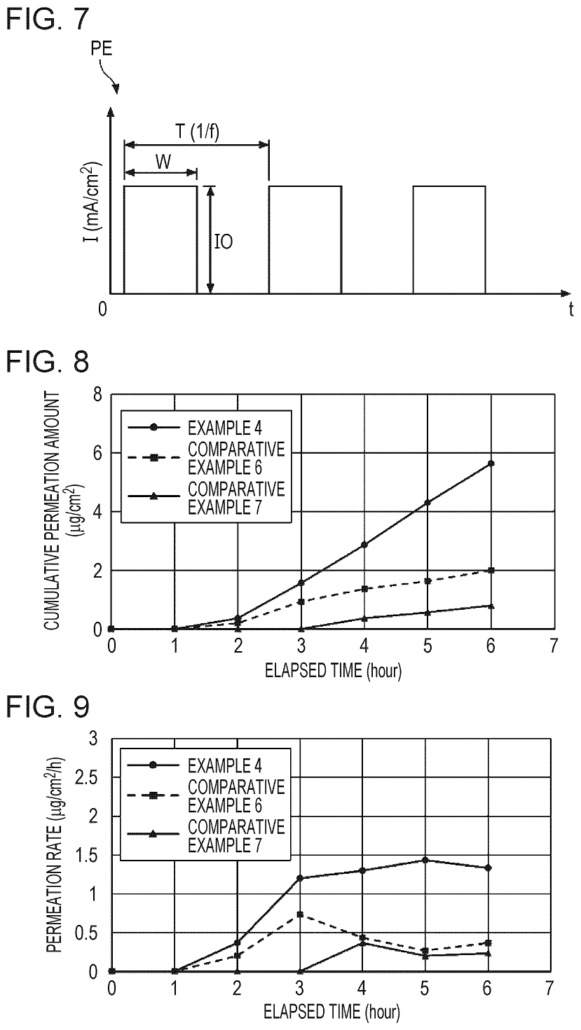

The control unit 50 controls the current applied to the skin 300 via the anode 21. In one or more embodiments, the control unit 50 performs control so that a pulse current having a pulse frequency of 0.5 Hz or more and 50 Hz or less is applied to the skin 300. The pulse frequency may be 0.8 Hz or more and 40 Hz or less, 1 Hz or more and 30 Hz or less, or 2 Hz or more and 25 Hz or less. Also, in one or more embodiments, the pulse current may have a pulse width of 10 ms or more and 1000 ms or less. The pulse width may be 20 ms or more and 700 ms or less, or 30 ms or more and 500 ms or less. In the present specification and claims, the pulse frequency refers to the number of continuous pulse currents in a predetermined period per second. The pulse current refers to a current applied in a predetermined period, in which a current with a peak flows during a period of time of a predetermined time width within one period. The pulse width refers to a predetermined time width (for example, a half-value width) in one period of the pulse current. The pulse current obtained by the control unit 50 will be described later in detail with reference to FIG. 7.

Here, the iontophoresis device 100 is not limited to the configuration in which the drug 31 is disposed between the anode 21 and the skin 300. For example, when the drug 31 is a negatively charged anion, the drug 31 may be disposed between the cathode 22 and the skin 300. With this arrangement, the iontophoresis device 100 uses the electrical repulsive force between the anion serving as the drug 31 and the cathode 22 to move the drug 31 disposed between the cathode 22 and the skin 300 in the direction where the skin 300 is located. Thus, the iontophoresis device 100 can supply the drug 31 into the body through the skin 300. In this arrangement, the control unit 50 controls the current applied to the skin 300 via the anode 21 and the cathode 22. In this arrangement, the cathode 22 corresponds to the electrode electrically connected to the power supply 10 and corresponds to the first electrode. The anode 21 corresponds to the second electrode. However, in iontophoresis, since electroosmotic flow, which is one of the driving forces for moving the drug 31, is a flow of a solvent from the anode 21 to the cathode 22, a configuration in which a positively charged cation serving as the drug 31 is disposed between the anode 21 and the skin 300 is advantageous.

### (Configuration of Control Unit 50)

FIG. 6 is a block diagram showing an example of the configuration of the control unit 50. As shown in FIG. 6, the control unit 50 includes a pulse generation circuit 51, a constant voltage control circuit 52, and a constant current element 53. The control unit 50 is, for example, a substrate on which these components are mounted. In one or more embodiments, the control unit 50 generates a pulse current PE by applying a pulse voltage PV subjected to constant voltage control by the constant voltage control circuit 52 to the constant current element 53. The control unit 50 applies the pulse voltage PV subjected to constant voltage control to the constant current element 53, thereby generating the pulse current PE having a substantially constant current density in an ON period with a simple circuit configuration.

The pulse generation circuit 51 is an electric circuit that generates a pulse signal PS that continues in a predetermined period and serves as a source for generating a pulse current. The pulse signal refers to a signal which is turned on only for a predetermined time width, that is, a pulse width, in one period and is turned off for a period of time other than the pulse width in one period.

The constant voltage control circuit 52 is an electric circuit that controls an input voltage to be output as a substantially constant voltage. Here, the constant voltage control circuit 52 controls the pulse signal PS generated by the pulse generation circuit 51 so that the voltage during the ON period is substantially constant. The constant voltage control circuit 52 applies the pulse voltage PV obtained as a result of the control to the constant current element 53.

The constant current element 53 is an element through which a substantially constant current can always flow. For example, a Zener diode can be used as the constant current element 53. The pulse current PE is applied to the skin 300 from the constant current element 53 via the anode 21.

For example, when the power supply 10 such as a coin-type lithium battery is attached to the iontophoresis device 100, the iontophoresis device 100 generates the pulse signal PS through the pulse generation circuit 51.

Subsequently, the iontophoresis device 100 applies, to the constant current element 53, the pulse voltage PV controlled by the constant voltage control circuit 52 so that the voltage in the ON period of the pulse signal PS input from the pulse generation circuit 51 is substantially constant.

Subsequently, the iontophoresis device 100 applies the pulse current PE from the constant current element 53 to the skin 300 via the anode 21. As described above, the iontophoresis device 100 can apply the pulse current PE to the skin 300.

In addition to the components shown in FIG. 6, the control unit 50 may include a switch for switching between starting and stopping of the iontophoresis device 100 by switching between ON and OFF of electric power supply from the power supply 10 to the control unit 50 in response to an operation by the operator. Since the control unit 50 includes the switch, electric power can be used only when necessary, and thus the iontophoresis device 100 can reduce power consumption.

The control unit 50 does not necessarily include the constant voltage control circuit 52 and the constant current element 53, and may apply, to the skin 300, a pulse current PE obtained by pulse-modulating the current input from the power supply 10 by the pulse generation circuit 51. However, the current density of the pulse current PE obtained by this configuration may vary due to changes in the voltage input from the power supply 10 and the load resistance, etc. Therefore, from the viewpoint of applying a pulse current PE having a substantially constant current density to the skin 300, it is preferable to use the configuration shown in FIG. 6.

Alternatively, the control unit 50 may include a constant current control circuit instead of the constant voltage control circuit 52 and the constant current element 53, and may apply a pulse current PE obtained by the constant current control circuit to the skin 300. However, the pulse current PE obtained by this configuration may not have a substantially constant current density depending on control delay or the like. Therefore, from the viewpoint of applying a pulse current PE having a substantially constant current density to the skin 300, it is preferable to use the configuration shown in FIG. 6.

Each function of the pulse generation circuit 51 and the constant voltage control circuit 52 in the control unit 50 may be realized by a processor implemented by an electronic circuit, an application specific integrated circuit (ASIC), a digital signal processor (DSP), a field-programmable gate array (FPGA), or a combination thereof.

### <One Example of Pulse Current PE>

FIG. 7 is a diagram illustrating an example of the pulse current PE applied to the skin 300 from the control unit 50 via the anode 21. FIG. 7 shows the variation of a current density I as a function of a time t.

In FIG. 7, the pulse current PE has a substantially rectangular waveform. In one or more embodiments, the pulse current PE has a pulse frequency f of 0.5 Hz or more and 50 Hz or less. Further, in one or more embodiments, the pulse current PE may have a pulse width W of 10 ms or more and 1000 ms or less. A period T is the period of the pulse current PE. A current density 10 is the current density during the period of time of the pulse width W in the pulse current PE. Further, a duty ratio D of the pulse current PE can be calculated by D=W/T×100. The waveform of the pulse current PE is not limited to a substantially rectangular waveform, and may be a substantially sinusoidal waveform, a substantially triangular waveform, or the like.

### EXAMPLES AND COMPARATIVE EXAMPLES

Hereinafter, Examples 1 to 4 and Comparative Examples 1 to 7 will be described. However, one or more embodiments of the present invention are not limited to these examples at all. In Examples and Comparative Examples, a current was applied to an attachment target from an iontophoresis device 100 attached to the attachment target under the conditions according to each example. Then, after the end of the current application, the skin irritability and the skin permeability of a drug in the attachment target were evaluated. Comparative Example 3 satisfies the conditions disclosed in Patent Literature 2. Comparative Examples 4 and 5 satisfy the conditions disclosed in Patent Literatures 1 and 3.

### (Skin Irritability Evaluation)

### (1) Evaluation Method

### Attachment Target

A test was conducted by attaching an iontophoresis device 100 having the following configuration to the skin of the inner side of the left or right forearm of the subject. Before attaching the iontophoresis device 100, the sites of the skin where the iontophoresis device 100 would be attached was wiped with absorbent cotton moistened with physiological saline.

### Configurations of Electrodes, First Reservoir 30, and Second Reservoir 40

Osaki Medical cross gauze cotton nonwoven fabrics cut into an approximately 2 cm×2 cm square shape were used as a first reservoir 30 and a second reservoir 40.

Electrocardiogram electrodes Red Dot 2360 manufactured by 3M Japan Limited and having an outer shape of approximately 3 cm×2 cm and a current-carrying area of 2 cm×2 cm=4 cm² were stacked thereon as electrodes and used. These electrodes correspond to an anode 21 and a cathode 22. Additionally, 0.3 mL of physiological saline was added to each of the first reservoir 30 and the second reservoir 40 as an electrolyte 41. Note that, in the skin irritability test, the first reservoir 30 and the second reservoir 40 without a drug 31 added were used.

### Power Supply 10 and Control Unit 50

A potentiogalvanostat PocketSTAT II manufactured by Ivium Technologies B.V. was used.

### Current Application Conditions

A constant current element 53 was connected in series between the anode 21 and the power supply 10 and control unit 50, and the anode 21 was brought into contact with the skin. The cathode 22 was connected to the power supply 10 and the control unit 50, and the cathode 22 was brought into contact with the skin. The current density was set to about 0.25 mA/cm² by setting the voltage from the power supply 10 and the control unit 50 to 10 V and controlling the amount of current applied to the skin from the constant current element 53 to about 1 mA. The current application time was 10 minutes in Examples 1 to 3 and Comparative Examples 2 to 5 in which a pulse current was applied, and was 5 minutes in Comparative Example 1 in which a non-pulse current, that is, a substantially constant current was applied, in order to match the total quantity of electricity. The waveform of the pulse current in Examples 1 to 3 and Comparative Examples 2 to 5 was a rectangular wave.

### Evaluation Criteria

After the current application was finished, the electrodes, the first reservoir 30, and the second reservoir 40 were peeled off from the skin, and the skin irritability after 5 minutes was visually evaluated based on the evaluation criteria shown in the following Table 1.

TABLE 1

| Points | Evaluation criteria |
|---|---|
| 5 | Edema and papules are present. |
| | Alternatively, there is strong erythema over the entire |
| | attachment sites. |
| 4 | Obvious erythema is observed over the entire attachment sites. |
| | Alternatively, erythema is observed in about 80% of the |
| | attachment sites. |
| 3 | Faint erythema is observed over the entire attachment sites. |
| | Alternatively, erythema is observed in about 50% of the |
| | attachment sites. |
| 2 | Slight erythema is observed over the entire attachment sites. |
| | Alternatively, erythema is observed in about 30% of the |
| | attachment sites. |
| 1 | No change. |

### (2) Evaluation Results

Table 2 shows a list of current application conditions and evaluation results for each example.

TABLE 2

| | | | | Comparative | Comparative | Comparative | Comparative | Comparative |
|---|---|---|---|---|---|---|---|---|
| Items | Example 1 | Example 2 | Example 3 | Example 1 | Example 2 | Example 3 | Example 4 | Example 5 |
| Pulse frequency | 0.5 | 5 | 50 | — | 0.005 | 0.05 | 100 | 1000 |
| f (Hz) | | | | | | | | |
| Period T (sec) | 2 | 0.2 | 0.02 | — | 200 | 20 | 0.01 | 0.001 |
| Pulse width W (sec) | 1 | 0.1 | 0.01 | — | 100 | 10 | 0.005 | 0.0005 |
| Duty ratio D (%) | 50 | 50 | 50 | — | 50 | 50 | 50 | 50 |
| Skin irritability | 2 | 1.5 | 2 | 5 | 5 | 4 | 3.5 | 4 |
| evaluation | | | | | | | | |

In Table 2, erythema was observed in 50% or more of the electrode attachment sites in Comparative Examples 1 to 5, and in particular, in Comparative Example 1, erythema appeared over the entire electrode attachment site on the cathode 22 side, and papules were also observed. In addition, in Comparative Example 2, strong erythema was observed over the entire electrode attachment sites of both the anode 21 and the cathode 22. On the other hand, in Examples 1 to 3, compared to Comparative Examples 1 to 5, the intensity of erythema was suppressed, the area of erythema was also reduced, and skin irritability was decreased. In particular, in Example 2, only very slight skin erythema was observed, and the “skin irritability evaluation” was “1.5”, which was an intermediate value between “1” and “2”, and showed the lowest value in this test.

### (Evaluation of Skin Permeability of Drug)

### (1) Evaluation Method

### Testing Device

The excised abdominal skin of a depilated male hairless rat (HWY/Slc, SPF, 5 weeks old) was punched into a 3.2 cm diameter circle and set in a vertical Franz cell. To the donor chamber of the Franz cell, 1.5 mL of a 0.5 wt % lanreotide acetate solution was added as a drug 31, and a zinc plate (0.5 cm×2 cm×0.5 mm) was immersed therein to form an anode 21. PBS was used as a receptor solution in the receptor chamber, and a silver/silver chloride rod (11 cm in length, 1 mm q in diameter) was inserted from the sampling port to form a cathode 22. The zinc plate and the silver/silver chloride rod were connected to the same power supply 10 and control unit 50 as those used in the skin irritability evaluation described above.

### Current Application Conditions

Example 4: A pulse current with a frequency of 5 Hz, a period of 100 ms, a duty ratio of 50%, a rectangular waveform, and a current density of 0.35 mA/cm² was applied.

Comparative Example 6: Instead of a pulse current, a current with a substantially constant current value and a current density of 0.35 mA/cm² was applied.

Comparative Example 7: No current was applied.

In order to match the total time of current application (total amount of applied current), the current was applied for 6 hours in Example 4 and for 3 hours in Comparative Example 6. Example 4 and Comparative Examples 6 to 7 were all conducted with n=3.

### Evaluation Items

Three items were evaluated: the cumulative amount of drug permeated, the permeation rate, and the relationship between the cumulative permeation amount and the quantity of electricity.

### Data Sampling

The receptor solution was sampled every hour, the amount of drug permeated through the skin was quantified by high-performance liquid chromatography (HPLC), and the above three items were evaluated with time. In Comparative Example 6, even after the end of the current application, the sampling was continued until 6 hours elapsed from the start of the test, as in Example 4 and Comparative Example 7. The HPLC measurement conditions are shown below.

### <HPLC Measurement Conditions>

HPLC system: high-performance liquid chromatograph (LC2010C) manufactured by Shimadzu Corporation

Column: ODS, 4.6 mm φ×15 cm, 5 μm

Column temperature: 40° C.

Mobile phase: A: 0.1% TFA aqueous solution, B: acetonitrile

Time program: shown in Table 3 below.

TABLE 3

| Time | Mobile phase A | Mobile phase B |
|---|---|---|
| (min) | (vol/%) | (vol/%) |
| 0 to 20 | 90→10 | 10→90 |
| 20 to 25 | 10 | 90 |
| 25 to 25.01 | 10→90 | 90→10 |
| 25.01 to 30 | 90 | 10 |

Detection wavelength: 220 nm

Flow rate: 1.0 mL/min

Lanreotide detection time: 7.5 min

### (2) Evaluation Results

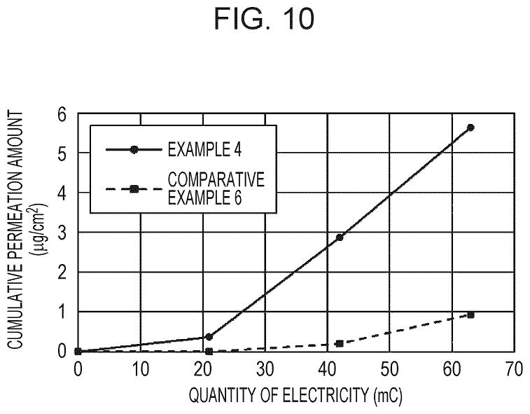

Evaluation results of skin permeability of the drug will be described with reference to FIGS. 8 to 10. FIG. 8 is a graph showing the cumulative amount of drug permeated. FIG. 9 is a graph showing the permeation rate of the drug. FIG. 10 is a graph showing the relationship between the cumulative amount of drug permeated and the quantity of electricity. In FIGS. 8 to 10, the black circular plots represent data of Example 4, the black square plots represent data of Comparative Example 6, and the black triangular plots represent data of Comparative Example 7.

As shown in FIGS. 8 and 9, the drug skin permeability was the highest in Example 4, followed by Comparative Example 6, and the lowest in Comparative Example 7. In FIG. 8, in Example 4 and Comparative Example 6, permeation of the drug through the skin was observed after about 2 hours from the start of the test, whereas in Comparative Example 7, permeation of the drug through the skin was not observed until 4 hours from the start of the test. Further, the cumulative amount of drug permeated in Example 4 was larger than that in Comparative Example 6 at any time after 2 hours from the start of the test at which skin permeation was first observed, and the difference increased with time. In addition, as shown in FIG. 9, the drug permeation rate of Example 4 was higher than those of Comparative Examples 6 and 7 at any time after 2 hours from the start of the test at which skin permeation was first observed. The drug permeation rate of Example 4 was maintained from 3 hours to 6 hours after the start of the test, whereas the drug permeation rate of Comparative Example 6 decreased from 4 hours after the start of the test.

As shown in FIG. 8, the cumulative permeation amount in Example 4 after 3 hours from the start of the test was larger than that in Comparative Example 6, although the amount of applied current was about half that in Comparative Example 6. In addition, as shown in FIG. 10, when the cumulative permeation amounts at the same quantity of electricity were compared, the cumulative permeation amount of Example 4 was clearly larger than the cumulative permeation amount of Comparative Example 6, and finally became 5 times or more.

From these results, it was found that the permeability of the drug was improved in Example 4 and Comparative Example 6 as compared with Comparative Example 7, and the permeation of the drug was promoted by the application of the current. Further, in Example 4 and Comparative Example 6, the permeability of the drug was improved in Example 4 as compared with Comparative Example 6, and it was found that the application of the pulse current further promoted the permeation of the drug as compared with the application of the constant current.

From the results of the evaluation of the skin irritability and the evaluation of the skin permeability of the drug described above, it was found that the skin permeability of the drug can be enhanced while reducing skin irritability by the application of the pulse current.

### <Main Effects of Iontophoresis Device 100>

As described above, in one or more embodiments, the control unit 50 performs control so that a pulse current PE having a pulse frequency f of 0.5 Hz or more and 50 Hz or less is applied to the skin 300. Thus, the iontophoresis device 100 can reduce the skin irritability due to the applied current while increasing the skin permeability of the drug 31. In addition, in one or more embodiments, the pulse current PE applied to the skin 300 may have a pulse width W of 10 ms or more and 1000 ms or less. This also allows the iontophoresis device 100 to reduce the skin irritability due to the applied current while increasing the skin permeability of the drug 31.

Further, in one or more embodiments, the iontophoresis device 100 includes the first reservoir 30 which is disposed between the skin 300 and the anode 21 and stores the drug 31, and the second reservoir 40 which is disposed between the skin 300 and the cathode 22 and stores the electrolyte 41. The control unit 50 controls the current applied to the skin 300 via the anode 21. According to this configuration, it is possible to promote electrical repulsion according to the applied current E and an action of the convective movement of water, and to increase skin permeability of the drug.

Although one or more embodiments of the present invention have been described in detail above, the present invention is not limited to these embodiments, and various modifications or changes can be made within the scope of the gist of one or more embodiments of the present invention described in the claims.

All the numerals such as ordinal numbers and quantities used in the description of the embodiments are examples for specifically describing the technique of one or more embodiments of the present invention, and the present invention is not limited to the exemplified numerals. In addition, the connection relationships between the constituent elements are examples for specifically describing the technique of one or more embodiments of the present invention, and the connection relationships for realizing the functions of one or more embodiments of the present invention are not limited thereto.

Aspects of one or more embodiments of the present invention are, for example, as follows.
<1> An iontophoresis device including a power supply; an electrode electrically connected to the power supply; a drug disposed between skin and the electrode; and a control unit configured to control a current applied to the skin via the electrode, wherein the control unit performs control so that a pulse current having a pulse frequency of 0.5 Hz or more and 50 Hz or less is applied to the skin.
<2> The iontophoresis device according to <1> above, wherein the pulse current has a pulse width of 10 ms or more and 1000 ms or less.
<3> An iontophoresis device including a power supply; an electrode electrically connected to the power supply; a drug disposed between skin and the electrode; and a control unit configured to control a current applied to the skin via the electrode, wherein the control unit performs control so that a pulse current having a pulse width of 10 ms or more and 1000 ms or less is applied to the skin.
<4> The iontophoresis device according to any one of <1> to <3> above, wherein the control unit generates the pulse current by applying a pulse voltage subjected to constant voltage control to a constant current element.
<5> The iontophoresis device according to any one of <1> or <4> above, wherein the electrode includes a pair of electrodes including a first electrode and a second electrode, the iontophoresis device includes a first reservoir that is disposed between the skin and the first electrode and stores the drug, and a second reservoir that is disposed between the skin and the second electrode and stores an electrolyte for moving ions, and the control unit controls the current applied to the skin via the first electrode.

This application claims the benefit of priority based on Japanese Patent Application No. 2023-055104, filed on Mar. 30, 2023. The entire disclosure of Japanese Patent Application No. 2023-055104 filed on Mar. 30, 2023 is incorporated herein by reference.

### REFERENCE SIGNS LIST

1 cover tape
2 base material
3 skin contact member
10 power supply
20 pair of electrodes
21 anode (one example of electrode, one example of first electrode)
22 cathode (one example of second electrode)
30 first reservoir
31 drug
40 second reservoir
41 electrolyte
50 control unit
51 pulse generation circuit
52 constant voltage control circuit
53 constant current element
60 wiring line
100 iontophoresis device
200 living body
300 skin
301 stratum corneum
302 epidermis
303 dermis
310 subcutaneous tissue
II region
CI counterion
D duty ratio
E applied current
f pulse frequency
I, I0 current density
PE pulse current
PS pulse signal
PV pulse voltage
t time
T period
W pulse width

Although the disclosure has been described with respect to only a limited number of embodiments, those skilled in the art, having benefit of this disclosure, will appreciate that various other embodiments may be devised without departing from the scope of the present disclosure. Accordingly, the scope of one or more embodiments of the invention should be limited only by the attached claims.

1. An iontophoresis device comprising:
an electrode attached to skin via a drug; and
a control unit that is connected to the electrode and configured to generate a pulse current having a frequency of 0.5 Hz or more and 50 Hz or less.

2. An iontophoresis device comprising:
a power supply;
an electrode electrically connected to the power supply;
a drug disposed between skin and the electrode; and
a control unit configured to control a current applied to the skin via the electrode,
wherein the control unit controls the current so that a pulse current having a pulse frequency of 0.5 Hz or more and 50 Hz or less is applied to the skin.

3. The iontophoresis device according to claim 1, wherein the pulse current has a pulse width of 10 ms or more and 1000 ms or less.

4. The iontophoresis device according to claim 2, wherein the pulse current has a pulse width of 10 ms or more and 1000 ms or less.

5. An iontophoresis device comprising:
an electrode attached to skin via a drug; and
a control unit that is connected to the electrode and configured to generate a pulse current having a pulse width of 10 ms or more and 1000 ms or less.

6. An iontophoresis device comprising:
a power supply;
an electrode electrically connected to the power supply;
a drug disposed between skin and the electrode; and
a control unit configured to control a current applied to the skin via the electrode,
wherein the control unit controls the current so that a pulse current having a pulse width of 10 ms or more and 1000 ms or less is applied to the skin.

7. The iontophoresis device according to claim 1, wherein the control unit generates the pulse current by applying a pulse voltage subjected to constant voltage control to a constant current element.

8. The iontophoresis device according to claim 2, wherein the control unit generates the pulse current by applying a pulse voltage subjected to constant voltage control to a constant current element.

9. The iontophoresis device according to claim 1, wherein:
the electrode comprises a pair of electrodes including a first electrode and a second electrode,
the iontophoresis device comprises a first reservoir that is disposed between the skin and the first electrode and stores the drug, and
a second reservoir that is disposed between the skin and the second electrode and stores an electrolyte for moving ions.

10. The iontophoresis device according to claim 2, wherein:
the electrode comprises a pair of electrodes comprising a first electrode and a second electrode,
the iontophoresis device comprises a first reservoir that is disposed between the skin and the first electrode and stores the drug,
a second reservoir that is disposed between the skin and the second electrode and stores an electrolyte for moving ions, and
the control unit controls the current applied to the skin via the first electrode.

11. The iontophoresis device according to claim 4, wherein:
the electrode comprises a pair of electrodes including a first electrode and a second electrode,
the iontophoresis device comprises a first reservoir that is disposed between the skin and the first electrode and stores the drug,
a second reservoir that is disposed between the skin and the second electrode and stores an electrolyte for moving ions, and
the control unit controls the current applied to the skin via the first electrode.
